(19) United States
(12) Patent Application Publication
(10) Pub. No.: US 2006/0002899 A1
(43) Pub. Date: Jan. 5, 2006

(54) Generation of dendritic cells from cd34+precursors

(76) Inventors: Alison Mary Rice, Queensland (AU); Derek Hart, Queensland (AU); Slavica Vuckovic, Queensland (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/525,928

(22) PCT Filed: Aug. 29, 2003
(86) PCT No.: PCT/AU03/01113

§ 371 Date: Jul. 26, 2005

(30) Foreign Application Priority Data
Aug. 30, 2002 (AU) 2002951082

(51) Int. Cl. A61K 45/00; C12N 5/08

(52) U.S. Cl. 424/93.7; 435/372

(57) ABSTRACT

The present invention relates to a method for inducing development of dendritic cells from CD34⁺ precursor cells. More particularly, the present invention relates to a method of differentiating CD34⁺ precursor cells into myeloid- and or lymphoid-dendritic cells. The present invention provides a protocol for the development of dendritic cells from a biological sample inter alia cord blood, bone marrow or peripheral blood CD34⁺ stem cells. The dendritic cells of the present invention are useful as therapeutic cellular agents such as in the development of vaccines and in modulating immunological responsiveness. More particularly, the present invention provides compositions which can be used for inducing a protective immune response in a subject against inter alia pathogenic infections, autoimmune diseases and cancer.

### BACKGROUND OF THE INVENTION

1. Field of the Invention

The present invention relates to a method for inducing development of dendritic cells from CD34⁺ precursor cells. More particularly, the present invention relates to a method of differentiating and expanding CD34⁺ precursor cells into myeloid- and or lymphoid-dendritic cells. The present invention provides a protocol for the development of dendritic cells from a biological sample inter alia cord blood, bone marrow or peripheral blood CD34⁺ stem cells. The dendritic cells of the present invention are useful as therapeutic cellular agents such as in the development of vaccines and in modulating immunological responsiveness. More particularly, the present invention provides methods for inducing a protective immune response in a subject against inter alia pathogenic infections, autoimmune diseases and cancer using compositions comprising antigen-presenting dendritic cells.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Dendritic cells (DC) are potent cellular activators of primary immune responses (Hart, Blood 90: 3245-3287, 1997). Immature myeloid DC in non-lymphoid organs react to endocytose and process antigens and migrate via blood and lymph to T cell areas of lymphoid organs. Here, the mature cells present foreign peptide complexed to MHC Class I and II to T cells and deliver unique signals for T-cell activation (immuno-stimulation). They also stimulate B lymphocytes and NK cells. DC undergo differentiation /activation during this process, lose their antigen-capturing capacity and become mature, immuno-stimulatory DC that trigger naive T-cells recirculating through the lymphoid organs. The lymphoid DC subset may have a different migration pathway and although capable of stimulating allogeneic and autologous T-lymphocytes they have been suggested to have a regulatory function (Grouard et al., J. Exp. Med. 185: 1101-1111, 1997). As part of the differentiation/activation process, DCs up-regulate certain relatively selectively-expressed cell surface molecules such as the CMRF-44 and CD83 antigens. DC in the thymus and blood that do not have an activated co-stimulating phenotype probably contribute to central and peripheral tolerance.

Blood dendritic cells (BDC) are released from bone marrow into the peripheral blood before homing to the tissues as surveillance DC for the immune system. The two major subsets of BDC are myeloid DC (CD11cu⁺ CD123^{-/dim}) and lymphoid (CD123^{hi} CD11c⁻).

There is a need to develop a protocol for generating BDC and in particular myeloid BDC and lymphoid BDC sub-populations. Such cells are useful as potential cellular agents for use, for example, in the manufacture of vaccines or in modulating immunological responsiveness. However, DC circulate in low number in the peripheral blood system and are, hence, difficult to isolate. A protocol is required, therefore, to generate a ready source of BDC.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention provides a method for inducing or otherwise facilitating development of blood dendritic cells (BDC) from CD34⁺ precursor cells. Generally, the CD34⁺ precursor cells are from sources such as cord blood, bone marrow or peripheral blood. Cord blood is particularly preferred. The protocol generally involves sorting CD34⁺ precursor cells into a-myeloid population and/or a lymphoid population. The myeloid population is generally CD33⁺CD7⁻CD10⁻ and the lymphoid population is generally CD33⁺CD7⁺CD10⁺. One or both sub-populations of CD34⁺ precursor cells are then cultured into the presence of one or more cytokines and preferably a cocktail of cytokines for a time and under conditions sufficient for CD34⁺-derived DC to develop. The myeloid DC precursors differentiate via either CD14 or CD1a pathways. Within the expanded population, monocytes (CD14⁺), granulocytes (CD15⁺), myeloid BDC-like cells (CD11c⁺CD123⁻) and lymphoid BDC-like cells (CD11c⁻CD123^{hi}) may develop. The latter myeloid and lymphoid BDC are proposed to be potential therapeutic cellular agents for the development of vaccines and to modulate immunological responsiveness.

The present invention provides, therefore, a method for generating myeloid- or lymphoid-like BDC, said method comprising isolating CD34⁺ precursor cells, sorting into a myeloid and/or lymphoid population and culturing either or both populations in the presence of one or more cytokines or functional, recombinant or chemical homologs or equivalents thereof for a time and under conditions sufficient for CD34⁺ cell expansion to occur and then isolating the CD34⁺-derived myeloid- or lymphoid-like BDC.

The useful cytokines are, for example, flt3, SCF, IL-3, IL-6, GM-CSF, G-CSF and/or TNFα.

The ability to enrich for, or generate, myeloid- or lymphoid-like BDC permits a marked improvement over monocyte-derived DC. The isolated cells may be used to generate vaccines to induce an immunological response against specific antigens or may be used to induce immunological tolerance or non-responsiveness.

The present invention provides, therefore, an isolated population of lymphoid- or myeloid-like BDC which present a peptide on their cell surface in the context of an MHC molecule. These antigen-presenting dendritic cells can then be used in vaccine development or as potential therapeutic cellular agents to, for example, induce immunological tolerance or non-responsiveness or induce protective immune responses against cancers or pathogenic agents.

The present invention further contemplates the use of myeloid- or lymphoid-like BDC derived from CD34⁺ precursor cells in the manufacture of a population of potential therapeutic cellular agents.

The present invention also provides vaccines comprising the isolated myeloid- or lymphoid-like BDC loaded with particular antigens.

Reference to a “CD⁻” includes a “CD^{lo}” or “CD^{-/-}” marker.

### BRIEF DESCRIPTION OF THE FIGURES

FIG. 1 is a graphical representation showing the growth of cord blood (CB) CD34⁺ myeloid precursors in the presence of flt3, SCF, IL-3 and IL-6 to generate an expanded culture.

FIG. 2 is a graphical representation showing the emergence of CD14⁺ progeny.

FIG. 3 is a graphical representation showing the emergence of CD15⁺ progeny.

FIG. 4 is a graphical representation showing the emergence of CD14⁻ CD15⁻ progeny.

FIGS. 5(A)-(E) are graphical representations of CD11c⁺DC's existing in a CD14⁻-CD15⁻ population after precursor cell expansion.

FIG. 6 is a graphical representation showing that CD11c⁺HLA-DR⁺CD123⁻CD1a⁻ cells can induce a potential mixed lymphocyte reaction (MLR).

### DETAILED DESCRIPTION OF THE PREFERRED EMBODIMENTS

The present invention provides a protocol for developing myeloid- or lymphoid-like BDC form CD34⁺ precursor cells. Reference herein to “myeloid-like BDC” and “lymphoid-like BDC” includes myeloid BDC and lymphoid BDC, respectively.

Myeloid-like BDC have the potential for inducing more potent allogenic T-lymphocyte responses compared to granulocytes or monocytes and, hence, are proposed to be useful therapeutic cellular agents in the development of vaccines and to modulate immunological responsiveness.

Accordingly, one aspect of the present invention contemplates a method for generating and expanding a population of dendritic cells, said method comprising obtaining a population or source of CD34⁺ precursor cells, sorting this population into CD34⁺ precursor cells, culturing the population with one or more cytokines for a time and under conditions sufficient to obtain a CD34⁺-derived cell expansion culture and then isolating said dendritic cells from the expanded population.

In a related embodiment, the CD34⁻ precursor cell is cultured with one or more cytokines for a time and under conditions sufficient to induce differentiation and/or expansion into a specific lineage of dendritic cells. The specific lineages of dendritic cells contemplated by the methods of the present invention include, without being limited to, Langerhans cells, interstitial dendritic cells, afferent lymph veiled cells, blood dendritic cells and interdigitating cells.

In a preferred aspect, the present invention contemplates a method for generating a population myeloid- or lymphoid-like BDC, wherein the method comprises obtaining a population of CD34⁺ precursor cells, sorting this population into myeloid and/or lymphoid precursors, culturing either or both with one or more cytokines for a time and under conditions sufficient to obtain a CD34⁺-dervied cell expansion culture and then isolating myeloid-like BDC or lymphoid-like BDC from the expanded culture.

Reference to a “biological sample” should be understood to reference any sample which contains CD34⁺ precursor cells. In this regard, the biological sample may be derivable from both human and non-human organisms. Non-human organisms contemplated by the present invention include primates, livestock animals (e.g. sheep, pigs, cows, horses, donkeys), laboratory test animals (e.g. mice, hamsters, rabbits, rats, guinea pigs), domestic companion animals (e.g. dogs, cats), birds (e.g. chicken, geese, ducks and other poultry birds, game birds, emus, ostriches), captive wild or tamed animals (e.g. foxes, kangaroos, dingoes), reptiles and fish.

It should be understood that the biological sample may be any sample derived from the organism which contains CD34⁺ precursor cells. This includes reference to both samples which are naturally present in the organism, such as tissue and body fluids in a mammal (for example biopsy specimens such as lymphoid specimens, blood, lymph fluid or bronchial secretions) and samples which are introduced into the body of the organism and subsequently removed, such as, for example, the saline solution extracted from the lung fluid following a lung lavage.

CD34⁺ precursor cells may be isolated directly from the biological sample or the sample may require some processing before the cells can be isolated. For example, a biopsy sample may require homogenisation prior to testing. CD34⁺ precursor cells may be isolated directly from blood using, for example using immunomagnetic beads coated with anti-CD34 antibodies, or the blood samples may be processed first to isolate the cellular compartment of the blood, e.g. the PBMC, prior to the CD34⁺ faction being further purified from the PBMC.

The CD34⁺ precursor cells are preferably CD34⁺ precursor cells from any convenient source. The most convenient source is cord blood. Other sources include bone marrow and peripheral blood. Yet other sources of CD34⁺ precursor cells include,.stem cells, monocytes, amniotic fluid, chrionic villus and tissues.

In relation to these preferred sources of CD34⁺ precursor cells, another aspect of the present invention provides a method for generating a population of myeloid- or lymphoid-like BDC, said method comprising obtaining a population or source of CD34⁺ stem cells from cord blood, bone marrow and/or peripheral blood sorting this population into myeloid and/or lymphoid precursors, culturing either or both populations with one or more cytokines for a time and under conditions sufficient to obtain a CD34⁺-derived cell expansion culture and then isolating myeloid-like BDC or lymphoid-like BDC from the expanded cell culture.

By “obtaining” CD34⁺ precursor cells including stem cells means enriching, selecting and/or isolating CD34⁺ cells from mixed populations of cells. The CD34⁺ precursor cell culture does not have to be pure or solely or substantially CD34⁺ cells but a substantially homologous population of CD34⁺ cells is certainly preferred. The present invention extends, however, to heterogenous mixtures of cells provided the mixture comprises CD34⁺ precursor-cells and in particular CD34⁺ stem cells. Sorting of the CD34⁺ precursor cells provides a population of myeloid precursors having characteristic markers CD33⁺CD7⁻CD10⁻ and a population of CD33^{±}CD7⁺CD10⁺ lymphoid precursors. Either or both populations may then be subject to expansion-enhancing conditions. Although either myeloid or lymphoid precursor CD34⁺ cells may be employed in the practice of the present invention, up to the present time, myeloid precursor cells are particularly preferred. Myeloid precursor CD34⁺ cells give rise to myeloid-like BDC which are CD11c⁺CD123⁻ cells. Lymphoid precursor CD34⁺ cells have the potential to give rise to lymphoid-like BDC which are CD11c⁻CD123^{hi}. Conveniently, CD34⁺ cells may be collected by any convenient means such as being immobilized to magnetic particles or FACS sorting microspheres.

The cells may be isolated directly from a biological sample, for example using immunomagnetic beads. Alternatively, the sample may be manipulated first to facilitate the isolation of the CD34⁺ precursor cells.

Sorting is preferably conducted with a flow cytometer which comprises a “fluorescence-activated cell sorter” (FACS) [see, for example, “Methods in Cell Biology” Vol. 33, Darzynkiewica, Z. and Crissman, H. A., eds., Academic Press) and Dangl and Herzenberg, J. Immunol. Methods 52: 1-14, 1982].

Accordingly, in a preferred embodiment, the present invention contemplates a method for generating a population of myeloid-like BDC, said method comprising obtaining a population or source of CD34⁺ precursor cells, sorting this population to obtain myeloid precursor cells characterized by being CD33⁺CD7⁻CD10⁻, culturing this population with one or more cytokines for a time and under conditions sufficient to obtain a CD34⁺-derived cell expansion culture and then isolating myeloid-like BDC characterized by being CD11c⁺CD123⁻ from the expanded cell culture.

Preferably, the CD34⁺ precursor cells are derived or obtained from cord blood. However, precursor cells from bone marrow and/or peripheral blood are also contemplated by the present invention.

The CD34⁺ cell expansion conditions include incubating or culturing the cells in the presence of one or more cytokines. Preferably, a cocktail of two or more cytokines is employed. Cytokine employed by the methods of the present invention, include, without being limited to, flt3, SCF, IL-3, IL-6, GM-CSF, G-CSF, TNF-α, IL-4, TNF-β, LT-β, IL-2, IL-7, IL-9, IL-15, IL-13, IL-5, IL-1α, IL-1β, IFN-γ, IL-10, IL-17, IL-16, IL-18, HGF, IL-11, MSP, FasL, TRAIL, TRANCE, LIGHT, TWEAK, CD27L, CD30L, CD40L, APRIL, TALL-1, 4-1BBL, OX40L, GITRL, IGF-I, IGF-II, HGF, MSP, FGF-a, FGF-b, FGF-3-19, NGF, BDNF, NTs, Tpo, Epo, Ang1-4, PDGF-AA, PDGF-BB, VEGF-A, VEGF-B, VEGF-C, VEGF-D, PIGF, EGF, TGF-α, AR, BTC, HRGs, HB-EGF, SMDF, OB, CT-1, CNTF, OSM, SCF, Flt-3L, M-CSF, MK and PTN or their functional, recombinant or chemical equivalents or homologues thereof. Although a range of cytokines may be employed, particularly useful cytokines are those selected from: flt3, SCF, IL-3 and IL-6 or their functional, recombinant or chemical equivalents or homologs. Other useful cytokines include GM-CSF, G-CSF and TNFα.

Accordingly, another aspect of the present invention contemplates a method for generating a population of myeloid- or lymphoid-like BDC, said method comprising obtaining a population or source of CD34⁺ precursor cells, sorting this population into myeloid and/or lymphoid precursors, culturing either or both populations with one or more cytokines selected from flt3, SCF, IL-3, IL-6, GM-CSF, G-CSF and TNFα or their functional, recombinant or chemical equivalents or homologs for a time and under conditions sufficient to obtain a CD34⁺-derived cell expansion culture and then isolating myeloid-like BDC or lymphoid-like BDC from the expanded cell culture.

Preferably, the CD34⁺ precursor cells are CD34⁺ cord blood-derived stem cells. Even more preferably, the cells generated are myeloid-like BDC characterized by being CD11c⁺CD123⁻.

A range of cytokines or functional equivalents may be employed and the present invention extends to any and all conditions which facilitate cell expansion. In a most preferred embodiment, however, a cocktail of cytokines comprising flt3, SCF, IL-3 and IL-6 is employed.

Accordingly, another aspect of the present invention contemplates a method for generating a population of myeloid- or lymphoid-like BDC, said method comprising obtaining a population or source of CD34⁺ precursor cells, sorting this population into myeloid and/or lymphoid precursors, culturing either or both populations with a mixture of cytokines comprising flt3, SCF, IL-3 and IL-6 or their functional, recombinant or chemical equivalents or homologs of any or all of the above for a time and under conditions sufficient to obtain a CD34⁺-derived cell expansion culture and then isolating myeloid-like BDC or lymphoid-like BDC from the expanded cell culture.

In another preferred embodiment, the present invention provides a method for generating a population of myeloid- or lymphoid-like BDC, said method comprising obtaining a population or source of CD34⁺ stem cells from cord blood, bone marrow and/or peripheral blood, sorting this population into myeloid and/or lymphoid precursors, culturing either or both populations with a mixture of cytokines comprising flt3, SCF, IL-3 and IL-6 and optionally one or more of GM-CSF, G-CSF and TNFα or their functional, recombinant or chemical equivalents or homologs of any or all of the above for a time and under conditions sufficient to obtain a CD34⁺-derived cell expansion culture and then isolating myeloid-like BDC or lymphoid-like BDC from the expanded cell culture.

In yet another embodiment, the present invention contemplates a method for generating a population of myeloid-like BDC, said method comprising obtaining a population or source of CD34⁺ precursor cells, sorting this population to obtain myeloid precursor cells characterized by being CD33⁺CD7⁻CD10⁻, culturing both populations with a mixture of cytokines comprising flt3, SCF, IL-3 and IL-6 and optionally one or more of GM-CSF, G-CSF and TNFα or their functional, recombinant or chemical equivalents or homologs of any or all of the above for a time and under conditions sufficient to obtain a CD34⁺-derived cell expansion culture and then isolating myeloid-like BDC characterized by being CD11c⁺CD123⁻ from the expanded cell culture.

The myeloid- and lymphoid-like BDC populations obtainable by the method of the present invention are proposed to be useful in the generation of vaccines and to modulate immunological responsiveness.

The present invention provides, therefore, a population of cells comprising myeloid- or lymphoid-like BDC, said population of cells isolated or enriched from an expanded culture of CD34⁺ cells generated from myeloid or lymphoid precursor cells sorted from a population of CD34⁺ precursor cells and cultured in the presence of one or more cytokines.

Reference to a “population” includes reference to an isolated culture comprising a homogenous, a substantially homogenous or a heterogenous culture of cells. Preferably, the population of myeloid-or lymphoid-like cells is substantially homogenous for one or either of these types of cells. Generally, a “population” may also be regarded as an “isolated” culture of cells.

In a preferred embodiment, a cocktail of cytokines is used comprising two or more of flt3, SCF, IL-3 and/or IL-6 or functional, recombinant or chemical equivalents thereof. Even more preferably, a cocktail comprises at least all of the flt3 ligand, SCF, IL-3 and IL-6.

In a particularly preferred embodiment, the present invention further extends to a population of myeloid-like BDCs, said population generated by the method of obtaining a population or source of CD34⁺ precursor cells, sorting this population into myeloid and/or lymphoid precursors, culturing either or both populations with one or more cytokines selected from flt3, SCF, IL-3 and IL-6 and optionally one or more of GM-CSF, G-CSF n TNFα or their functional, recombinant or chemical equivalents or homologs for a time and under conditions sufficient to obtain a CD34⁺-derived cell expansion culture and then isolating myeloid-like BDC or lymphoid-like BDC from the expanded cell culture.

The isolated, population of myeloid- and/or lymphoid-like cells are useful in the manufacture of vaccines. In one embodiment, the cells are exposed, incubated or cultured with one or more antigens for a time and under conditions for the antigen to be taken up by the cells and presented in the context of peptide associated with an MHC molecule expressed on the cells surface. In one aspect of the present invention, the peptide is presented in the context of an MHC class I molecule present on the surface of the dendritic cell, wherein the antigen-presenting dendritic cell specifically targets CD8⁺-cytotoxic T lymphocytes for stimulation and subsequent expansion of a CD8⁺-specific T cell immune response. Alternatively, the peptide is presented in the context of an MHC class II molecule on the dendritic cell surface, wherein the antigen-presenting dendritic cell specifically targets CD4⁺ T helper cells resulting in the stimulation and expansion of a CD4-specific T cell immune response. In a most preferred embodiment, the dendritic cell presents peptides from a specific protein or organism in the context of both MHC class I and MFIC class II molecules. Co-expression in this context provides for the generation of both a CD4⁺ and CD8⁺ T cell response specific for the antigen of interest. Such a dual response is preferred in the establishment of a protective immune response.

Peptides presented in conjunction with the MHC molecules may be loaded either by incubating the dendritic cells with peptide fragments derived from the antigen of interest which are able to bind directly to the MHC molecule, e.g. such as a synthetically produced peptide comprising a minimal epitope. Methods for the generation of such peptides will be appreciated by one of skill in the art. Alternatively, polypeptides, entire proteins, cells, or fragments thereof may be mixed with the dendritic cells or precursors to the dendritic cells. These molecules can then be internalised by the dendritic cells, wherein the proteins/polypeptides are internally processed and eventually presented on the dendritic cell surface in the contest of either a class I or class II MHC molecule.

Alternatively, the antigen-presenting dendritic cell may be transfected with a polynucleotide encoding a polypeptide (or portion or other variant thereof) such that the polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets the antigen-presenting dendritic cell may be administered to a subject, resulting in transfection that occurs in vivo. In vivo and ex vivo transfection of dendritic cells, for example, may generally be performed using any methods known in the art, such as those described in WO 97/24447, or the gene gun approach described by Mahvi et al., 1997, Immunology and Cell Biology 75.456-460. Antigen loading of dendritic cells may be achieved by incubating dendritic cells or the CD34⁺ precursor cells with a polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that aids in the expression of (e.g., a carrier molecule). Alternatively, a dendritic cell may be pulsed with a non-conjugated immunological partner, separately or in the presence of the polypeptide.

Upon re-introduction of the antigen-load myeloid- or lymphoid-like BDC, an immune response is generated against the antigens. These antigens may be derived from pathogenic, microorganisms, parasites, cancer cells or other sources.

Using the methods of the present invention, protective immune responses can be generated against foreign proteins and polypeptides, in addition to inducing tolerance to self-proteins. As used herein, “protective immunity” should be understood to reference the development of an immune resporise against a target antigen. Such responses can be measured using immunological assays, such as proliferation assays, cytotoxic T cell assays, including chromium release assays, assays which measure the production of cytokines, such as IFN-γ, TNF-α, and IL-2. The methods for performing such assays are well known to those experienced in the field of immunology and are described in Immunology (Roit, Brostoff, Male: 6^{th} Edition).

In one aspect, protective immunity is induced against a specific pathogen. Such pathogens include, without being limited to Viruses, bacteria, fungi, ectoparasites, mycoplasms, Archea, algae, oomycetes, slime molds, nematodes and amoebeae.

In a preferred aspect, the antigen-presenting dendritic cells generated using the methods of the present invention are specific for a virus selected from the group consisting of Human Immunodeficiency Virus (HIV), the human papilloma virus, Epstein-Barr virus, the polio virus, the rabies virus, the Ebola virus, the influenza virus, the encephalitis virus, smallpox virus, the rabies virus, the herpes viruses, the sendai virus, the respitory syncytial virus, the othromyxoviruses, the measles viruses, the vesicular stomatitis virus, visna virus and cytomegalovirus.

In a related aspect, the antigen-presenting dendritic cells generated using the methods of the present invention are specific for a fungi selected from the group consisting of Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epiderniophyton spp., Exophiala jeanselmei, Exserohilum spp., Fonsecaea compacta, Fonsecaea pedrosoi, Fusarium oxysporum, Fusarium solani, Geotrichum candidum, Histoplasnia capsulatum var. capsulatum, Histoplasnia capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotesiudina rosatii, Onychocola canadenzsis, Paracoccidioides brasiliensis, Phialophora verrucosa, Piedraia hortae, Piedra iahortae, Pityriasis versicolor, Pseudoallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus.

In yet another aspect, the antigen-presenting dendritic cells generated using the methods of the present invention are specific for a bacterial protein, wherein the bacterial protein is derived from a bacterium selected from the group consisting of Bacillus anthracis, Bordetella pertussis, Vibrio cholerae, Escherichia coli, Shigella dyseinteriae, Clostridium perfringens, Clostridium botulinum, Clostridium tetani, Corynebacterium diphtheriae, Pseudomonas aeruginosa, Bacillus anthracis, Bordetella pertussis, Staphylococcus aureus and Streptococcus pyogenes.

In yet another aspect, the antigen presenting dendritic cells of the present invention are specific in inducing immunity against a variety of cancers in a subject. The methods of the present invention contemplate providing protection against cancers selected from the group consisting of, without being limited to, ABL1 protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytorna, Ataxia-telangiectasia, Basal Cell Carcinoma (Skin), Bladder Cancer, Bone Cancers, Bowel cancer, Brain Stem Glioma, Brain and CNS Tumours, Breast Cancer, CNS tumours, Carcinoid Tumours, Cervical Cancer, Childhood Brain Tumours, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dernatofibrosarcoma-protuberans, Desmoplastic-Small-Round-Cell-Tumour, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumour, Genitourinary Cancers, Germ Cell Tumours, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kaposi's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphonia, Non-Hodgkin's Lymphoma, Male Breast Cancer, Malignant-Rhabdoid-Tumour-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumours, Pituitary Cancer, Polycythemia Vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumours, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia, Wilms' Tumour.

The present invention also contemplates methods for protecting a subject against autoimmune diseases. As used herein, reference to an autoimmune disease, includes inducing protection against any disease caused the immune system mistakenly attacking self molecules, targeting the cells, tissues, and organs of a person's own body. Potection against autoimmune diseases contemplated by the present invention include, without being limited to, those selected from the group consisting of Addisons Disease, Allergies, Anemia, Ankylosing Spondylitis, Arthritis, Celiac Disease, Crohns Disease, Diabetes, Endometriosis, Fibromyaigia, Graves Disease, Hashimotos Disease, Hypothyroidism, Immune Diseases, Lupus, Lymphoma, Meniere's Disease, Multiple Sclerosis, Oral Diseases, Osteoporosis, Pleunrsy, Psoriasis, Reiters Syndrome, Rheumatoid Arthritis, Sarcoidosis, Scleroderna, Sjogrens Syndrome, Thrush, Vitiligo, Alopecia Areata, Antiphospholipid Syndrome (APS), Behcet's Disease, Ulcerative Colitis, Goodpasture Syndrome, Graft Versus Host Disease, Guillain-Barre Syndrome, Multiple Sclerosis, Myasthenia Gravis, Myositis, Pemphigus Vulgaris, Primary Biliary Cirrhosis, Rheumatic Fever, Vasculitis and Wegener's Granulomatosis.

Vaccines are also useful in the treatment or prophylaxis of inflammatory bowel disease and to increase levels of immune responsiveness such as during stress (e.g. surgery) or chemotherapy.

Alternatively, the myeloid- or lymphoid-like BDC may be loaded with sub-optimal levels of antigen or loaded with a super dose which can result in the induction of immuno-tolerance or immuno-non-responsiveness.

Accordingly, another aspect of the present invention contemplates a method of vaccinating a subject against an antigen including a cell carrying the antigen, said method comprising loading a myeloid- or lymphoid-like BDC with an amount of said antigen which will induce an immune response wherein said myeloid- or lymphoid-like BDC or its parent is prepared by the method of generating a population of lymphoid- or myeloid-like BDC, said method comprising obtaining a population or source of CD34⁺ precursor cells, sorting this population into myeloid and/or lymphoid precursors, culturing either or both populations with one or more cytokines for a time and under conditions sufficient to obtain a CD34⁺-derived cell expansion culture and then isolating myeloid-like BDC or lymphoid-like BDC from the expanded cell culture.

Preferably, the CD34⁺ precursor cells are CD34⁺ stem cells from cord blood.

Preferably, the myeloid and/or lymphoid precursor cells are cultured in the presence of a cytokine selected from the list comprising flt3, SCF, IL-3 and IL-6. Even more preferably, the cells are cultured in a cocktail of cytokines comprising flt3-ligand, SCF, IL-3 and IL-6. Other cytokines in the cocktail may optionally include GM-CSF, G-CSF and TNFα.

In a most preferred embodiment, the present invention contemplates a method of vaccinating a subject against an antigen including a cell carrying the antigen, said method comprising loading a myeloid-like BDC with an amount of said antigen which will induce an immune response wherein said myeloid-like BDC or its parent is prepared by the method of generating a population of myeloid-like BDC by obtaining a population or source of CD34⁺ precursor cells, sorting this population into myeloid precursors, culturing this population with one or more cytokines for a time and under conditions sufficient to obtain a CD34⁺-derived cell expansion culture, loading the myeloid-like BDC with an antigen and then introducing the BDC to the subject. Generally, the subject is the source of the CD34⁺ precursor cells.

In another embodiment, as indicated above, the myeloid- or lymphoid-like BDC are loaded with sub-optimal or excess doses of antigen to induce immuno-tolerance or non-responsiveness.

The present invention provides, therefore, compositions for modulating the immune system or a response thereby, said composition comprising a myeloid- or lymphoid-like BDC generated by the method as hereinbefore described or are progeny cells of these generated cells. Such cells may be optionally loaded with antigen to induce a protective immune response or with sub- or over-optimum levels to induce immuno-tolerance or non-responsiveness.

Generally, although not exclusively, when used in therapy, autologous BDC are used relative to the subject.

The preferred subject is a human but the present invention extends to other primates, livestock animals (e.g. sheep, cows, horses, pigs, donkeys, goats), companion animals (e.g. dogs, cats) or laboratory test animals (e.g. mice, rabbits, guinea pigs, hamsters) and avian species (e.g. chickens, game birds or aviary birds).

The present invention further contemplates the use of myeloid- or lymphoid-like BDC generated as hereinbefore described in the manufacture of a vaccine or therapeutic cellular agent.

In addition to the antigen-presenting dendritic cells generated using the methods of the present invention, compositions used in the treatment of a subject may additionally comprise adjuvants. As-used herein, “adjuvant” includes those adjuvants commonly used in the art to facilitate the stimulation of an immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); AS-2 (Smith-Kline Beecham); QS-21 (Aquilla); M4PL or 3d-MPL (Corixa Corporation, Hamilton, Mont.); LEIF; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; muramyl tripeptide phosphatidyl ethanolamine or an immunostimulating complex, including cytokines (e.g., GM-CSF or interleukin-2, -7 or -12) and immunostimulatory DNA sequences. In some embodiments, such as with the use of a polynucleotide vaccine, an adjuvant such as a helper peptide or cytokine can be provided via a polynucleotide encoding the adjuvant.

Such a vaccine or therapeutic cellular agent is also regarded herein as a medicament.

In one embodiment, the vaccine or therapeutic cellular agent is useful for the treatment or prophylaxis of cancer infection by pathogenic microorganism, parasite or virus or to induce immuno-tolerance or non-responsiveness such as in the treatment or prophylaxis of autoimmune disease conditions.

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective amount.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases such as those contemplated by the present invention, the physician needs to evaluate the production of an immune response against the virus, progression of the disease, and any treatment-related toxicity.

The present invention is further described by the following non-limiting Examples.

### EXAMPLE 1

### Generation of Myeloid-Like BDC

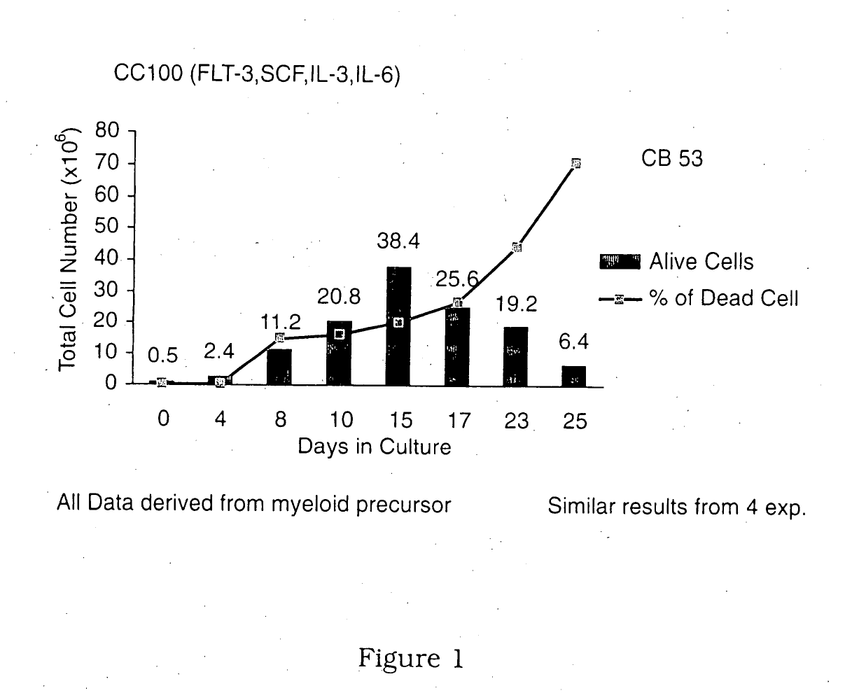

Sorted myeloid (CD33⁺CD7⁻CD10⁻) and lymphoid (CD34⁺CD33^{±}CD7⁺CD10⁺) precursors from enriched cord blood CD34⁺ cells were cultured in 24-well plates (4−5×10⁴ cells/ml) in H2000 serum free medium supplemented with a cocktail of cytokines (flt3-ligand 50 ng/ml, SCF 50 ng/ml, IL-3 10 ng/ml and IL-6 10 ng/ml) for 2-3 weeks. FIG. 1 shows the growth of cord blood CD34⁺ precursors in the presence of the cytokines. The progeny were assessed for phenotype on days 6-8 and every second day thereafter and also for their capacity to induce allogeneic T lymphocyte responses on days 8-12, of culture.

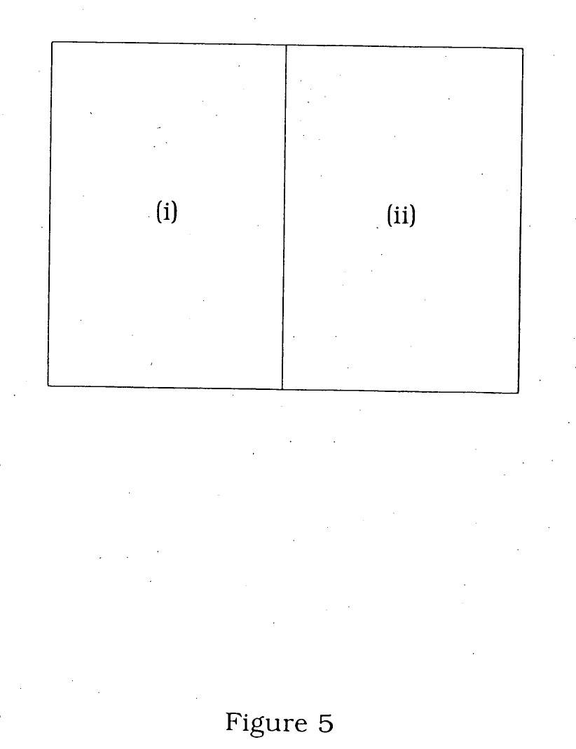

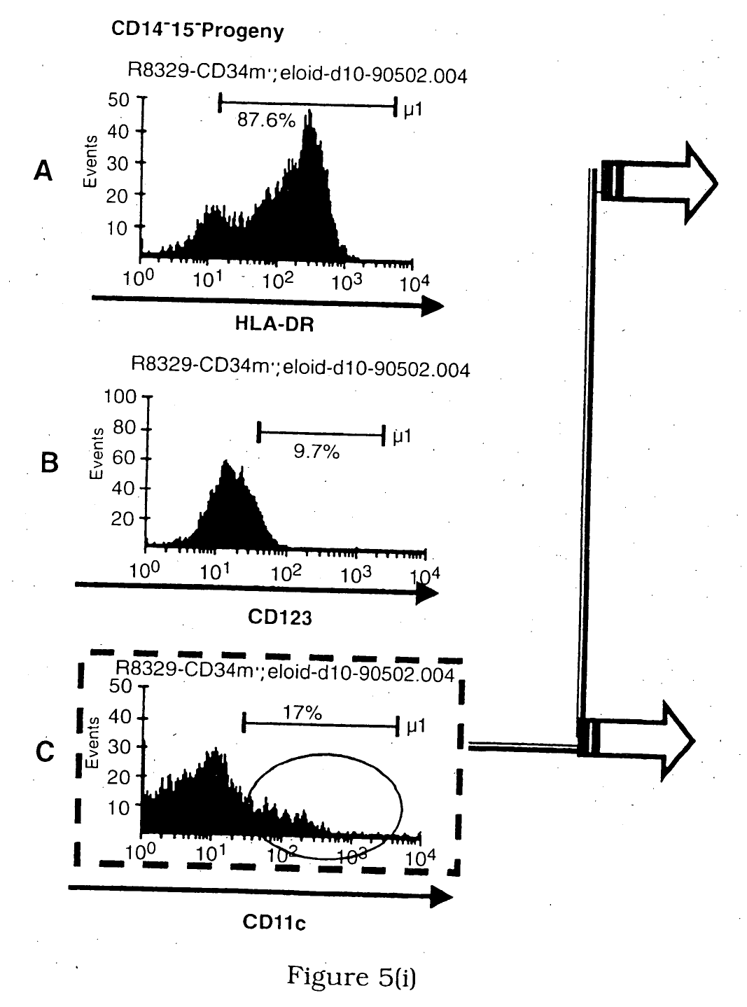

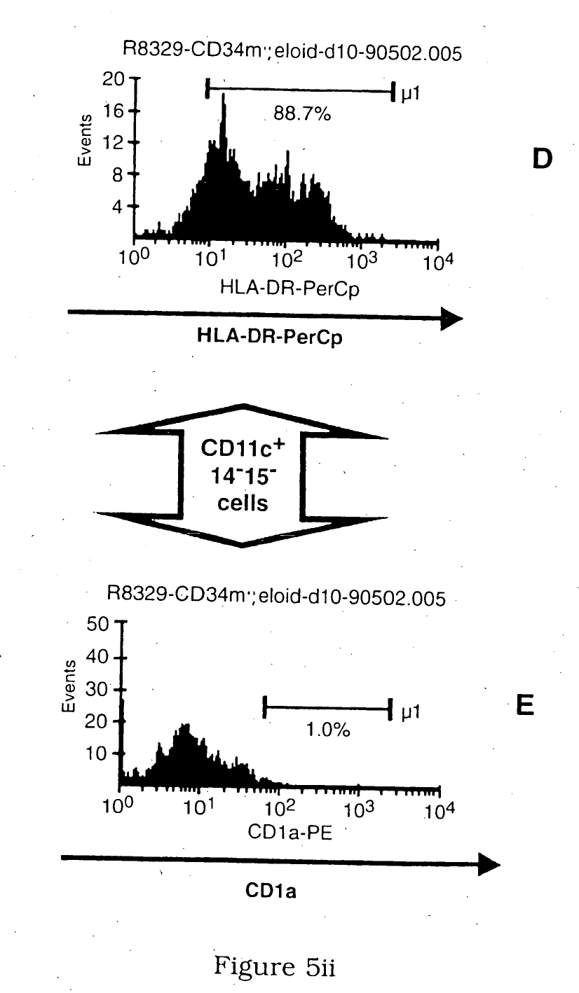

The presence of CD11c⁺ myeloid-like BDC in a CD14⁻CD15⁻ population is shown in FIGS. 5A-E.

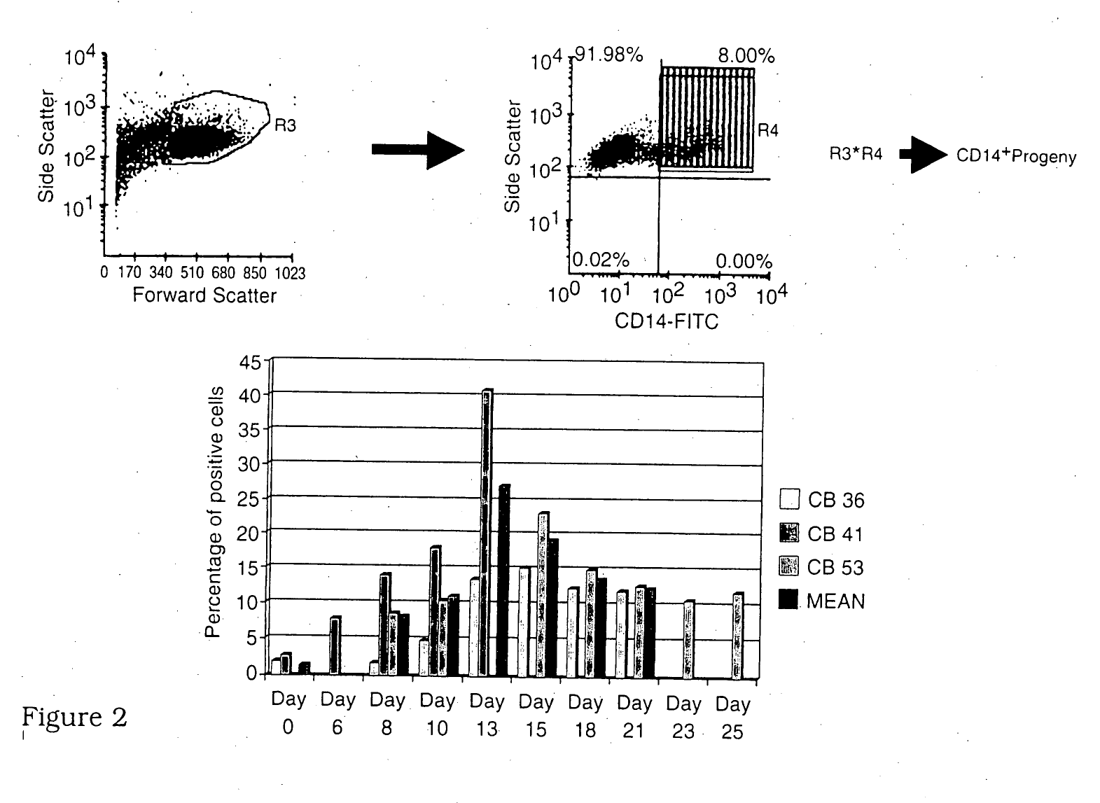

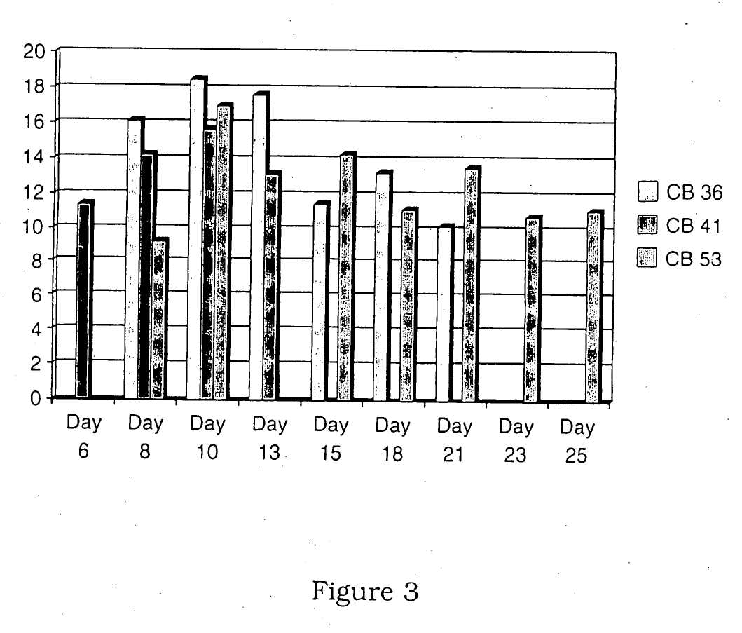

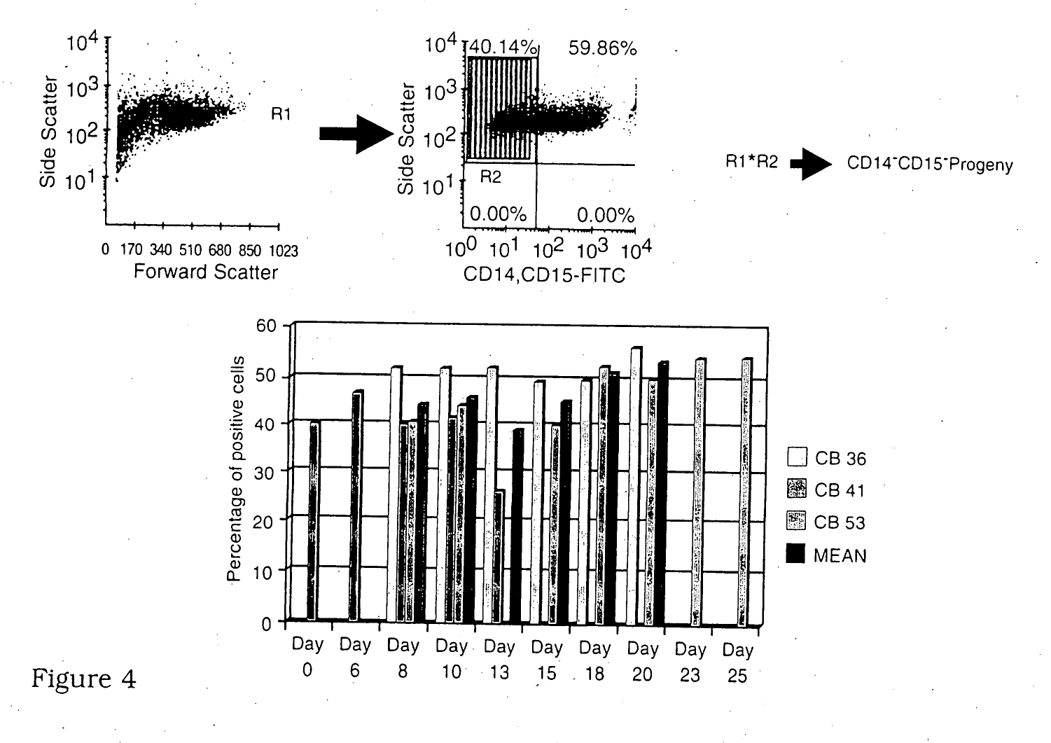

FIGS. 2-4 show the emergence of CD14⁺, CD15⁺ and CD14⁻CD15⁻ populations and

FIG. 5 shows the emergency of CD11c⁺CD14⁻CD15⁻ progeny.

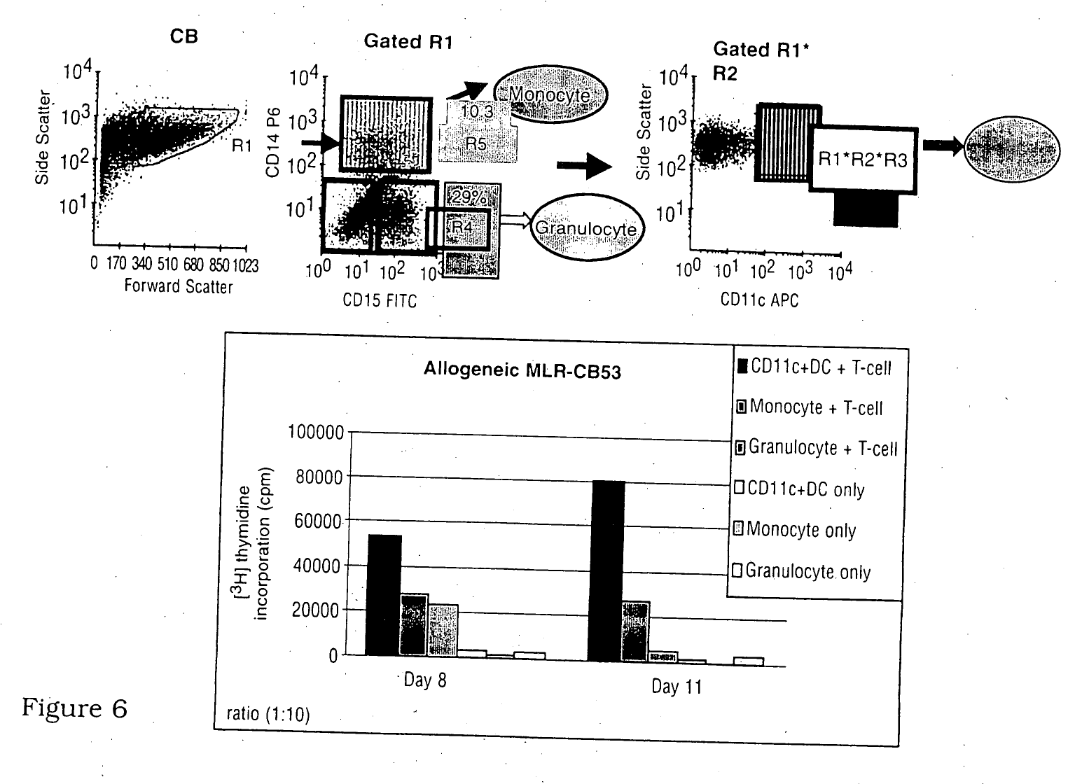

FIG. 6 shows CD11c⁺HLA-D⁺CD123⁻CD1a⁻ cells can induce a mixed lymphocyte reaction (MLR).

Peak CD34⁺ derived cell expansion was observed on day 10-13 of culture, resulting in a 70 to 100 fold increase in cell number. Thereafter, cell number decreased steadily, although viable cells (>60%) could be observed for up to 3 weeks. There.was no evidence of lineage differentiation during the initial period (4-6 days) of cell expansion. Monocytes (CD14⁺ cells), granulocytes (CD15⁺ cells), and myeloid-like BDC (CD11c⁺CD123⁻ cells) appeared subsequently (days 6-8) and were maintained during the period of culture. Only the CD11c⁺CD123⁻ progeny were capable of inducing potent allogeneic T lymphocyte responses, compare to monocytes and granulocytes.

### EXAMPLE 2

### Yield of CD34⁺, Myeloid/Lymphoid Precursors

The yield of CD34⁺, myeloid/lymphoid precursor is shown in Table 1.

TABLE 1

| | | | | Myeloid | Lymphoid |
|---|---|---|---|---|---|
| Sample | Volume | MNC | CD34⁺ | precursor | precursor |
| CB20 | 50 ml | 350 × 10⁶ | 5.8 × 10⁶ | 0.6 × 10⁶ | 0.12 × 10⁶ |
| CB53 | 50 ml | 300 × 10⁶ | 4 × 10⁶ | 0.5 × 10⁶ | 0.02 × 10⁶ |
| CB56 | 55 ml | 310 × 10⁶ | 3.5 × 10⁶ | 0.3 × 10⁶ | 0.04 × 10⁶ |
| CB30* | — | 360 × 10⁶ | 1.8-2.5 × 10⁶ | — | — |

Purity CD34⁺ cells: >98%
*Pranke, Acta Haematologica 105: 71-76, 2001

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Dangl and Herzenberg, J. Immunol. Methods 52: 1-14, 1982
Darzynkiewica, Z. and Crissman, H. A., eds., Academic Press) “Methods in Cell Biology” Vol. 33,
Grouard et al., J. Exp. Med. 185. 1101-1111, 1997.
Hart, Blood 90. 3245-3287, 1997.
Mahvi et al., Immunology and Cell Biology 75: 456-460, 1997.
Pranke, Acta Haematologica 105: 71-76, 2001
Roit, Brostoff, Male: 6^{th} Edition, Immunology

1. A method for generating a population of myeloid like blood dendritic cells or lymphoid like blood dendritic cells from CD34⁺ precursor cells said method comprising sorting said CD34⁺ precursor cells into a population of either myeloid precursor cells characterized by the markers CD33⁺, CD7⁻, CD10⁻ or a population of lymphoid precursor cells characterized by the markers CD34^{±}, CD7⁺, CD10⁺, culturing or expanding the myeloid or lymphoid precursor cells in the combination of Flt3, SCF, IL-3 and/or IL-6 for a time and under conditions sufficient for the myeloid precursor cells to give rise to the myeloid like blood dendritic cells characterized by being CD11c⁺ and CD123⁻ and the lymphoid precursor cells to give rise to lymphoid like blood dendritic cells characterized by being CD11c⁻, CD123^{hi}.

2. The method of any one of claim 1, wherein said CD34⁺ precursor cells are isolated from a biological sample selected from the group consisting of peripheral blood, PBCMs, stem cells, monocytes, amniotic fluid, chorionic villus, cord blood, and tissue.

3. The method according to any one of claims 1 or 2, wherein said population of CD34⁺ precursor cells is differentiated into a specific lineage of dendritic cells selected from the group consisting of myeloid dendritic cells, lymphoid dendritic cells, Langerhans cells, interstitial dendritic cells, Afferent lymph veiled cells, blood dendritic cells and interdigitating cells.

4. The method of claim 3, wherein said population of CD34⁺ precursor cell is differentiated into a heterogenous population of dendritic cells.

5. The method of claim 3, wherein said population of CD34⁺ precursor cells is differentiated into a substantially homogenous population of dendritic cells.

6. The method of claim 1, wherein the cytokine is selected from the group consisting of flt3, SCF, IL-3, IL-6, GM-CSF, G-CSF, TNF-α, IL-4, TNF-β, LT-β, IL-2, IL-7, IL-9, IL-15, IL-13, IL-5, IL-1α, IL-1β, IFN-γ, IL-10, IL-17, IL-16, IL-18, HGF, IL-11, MSP, FasL, TRAIL, TRANCE, LIGHT, TWEAK, CD27L, CD30L, CD40L, APRIL, TALL-1, 4-1BBL, OX40L, GITRL, IGF-I, IGF-II, HGF, MSP, FGF-a, FGF-b, FGF-3-19, NGF, BDNF, NTs, Tpo, Epo, Ang1-4, PDGF-AA, PDGF-BB, VEGF-A, VEGF-B, VEGF-C, VEGF-D, PIGF, EGF, TGF-α, AR, BTC, HRGs, HB-EGF, SMDF, OB, CT-1, CNTF, OSM, SCF, Flt-3L, M-CSF, MK and PTN or their functional, recombinant or chemical equivalents or homologues thereof.

7. The method of claim 6, wherein the cytokine is selected from the group consisting flt3, SCF, IL-3, IL-6, GM-CSF, G-CSF, TNF-α or their functional, recombinant or chemical equivalents or homologues thereof.

8. The method of claim 1, further comprising presenting a peptide on the surface of the dendritic cells, thereby providing a population of antigen presenting dendritic cells.

9. The method of claim 8, wherein said antigen presenting cell is capable of activating a population of T cells.

10. The method of claim 9, wherein said population of T cells occurs in vitro.

11. The method of claim 9, wherein said population of T cells occurs in vivo.

12. The method of any one of claims 9 to 11, wherein the T cell is selected from a population of CD4⁺ T cells and/or CD8⁺ T cells.

13. The method of claim 9, wherein said peptide is derived from a polypeptide isolated from a human protein, a pathogen protein or a protein derived from a cancer cell.

14. The method of claim 13, wherein said pathogen is selected from the group consisting of viruses, bacteria, fungi, ectoparasites, mycoplasms, Archea, algae, oomycetes, slime molds, nematodes and amoebeae.

15. The method of claim 14, wherein said virus is selected from the group consisting of Human Immunodeficiency Virus (HIV), the human papilloma virus, Epstein-Barr virus, the polio virus, the rabies virus, the Ebola virus, the influenza virus, the encephalitis virus, smallpox virus, the rabies virus, the herpes viruses, the sendai virus, the respitory syncytial virus, the othromyxoviruses, the measles viruses, the vesicular stomatitis virus, visna virus and cytomegalovirus.

16. The method of claim 14, wherein said fungi is selected from the group consisting of Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudoallesheria boydii, Pyrenochaeta romeroi, Rhizopus arrhizus, Scopulariopsis brevicaulis, Scytalidiutn dimidiatum, Sporothrix schenckii, Trichophyton spp., Trichosporon spp., Zygomcete fungi, Absidia corymbifera, Rhizomucor pusillus and Rhizopus arrhizus.

17. The method of claim 14, wherein is said bacteria are selected from the group consisting of Bacillus anthracis, Bordetella pertussis, Vibrio cholerae, Escherichia coli, Shigella dyseinteriae, Clostridium perfringens, Clostridium botulinum, Clostridium tetani, Corynebacterium diphtheriae, Pseudomonas aeruginosa, Bacillus anthracis, Bordetella pertussis, Staphylococcus aureus and Streptococcus pyogenes.

18. The method of claim 13, wherein said cancer cell is isolated from a cancer cell associated with a cancer selected from the group consisting of ABL1 protooncogene, AIDS Related Cancers, Acoustic Neuroma, Acute Lymphocytic Leukaemia, Acute Myeloid Leukaemia, Adenocystic carcinoma, Adrenocortical Cancer, Agnogenic myeloid metaplasia, Alopecia, Alveolar soft-part sarcoma, Anal cancer, Angiosarcoma, Aplastic Anaemia, Astrocytoma, Ataxia-telangiectasia, Basal Cell Carcinoma (Skin), Bladder Cancer, Bone Cancers, Bowel cancer, Brain Stem Glioma, Brain and CNS Tumours, Breast Cancer, CNS tumours, Carcinoid Tumours, Cervical Cancer, Childhood Brain Tumours, Childhood Cancer, Childhood Leukaemia, Childhood Soft Tissue Sarcoma, Chondrosarcoma, Choriocarcinoma, Chronic Lymphocytic Leukaemia, Chronic Myeloid Leukaemia, Colorectal Cancers, Cutaneous T-Cell Lymphoma, Dernatofibrosarcoma-protuberans, Desmoplastic-Small-Round-Cell-Tumour, Ductal Carcinoma, Endocrine Cancers, Endometrial Cancer, Ependymoma, Esophageal Cancer, Ewing's Sarcoma, Extra-Hepatic Bile Duct Cancer, Eye Cancer, Eye: Melanoma, Retinoblastoma, Fallopian Tube cancer, Fanconi Anaemia, Fibrosarcoma, Gall Bladder Cancer, Gastric Cancer, Gastrointestinal Cancers, Gastrointestinal-Carcinoid-Tumour, Genitourinary Cancers, Germ Cell Tumours, Gestational-Trophoblastic-Disease, Glioma, Gynaecological Cancers, Haematological Malignancies, Hairy Cell Leukaemia, Head and Neck Cancer, Hepatocellular Cancer, Hereditary Breast Cancer, Histiocytosis, Hodgkin's Disease, Human Papillomavirus, Hydatidiform mole, Hypercalcemia, Hypopharynx Cancer, IntraOcular Melanoma, Islet cell cancer, Kaposi's sarcoma, Kidney Cancer, Langerhan's-Cell-Histiocytosis, Laryngeal Cancer, Leiomyosarcoma, Leukaemia, Li-Fraumeni Syndrome, Lip Cancer, Liposarcoma, Liver Cancer, Lung Cancer, Lymphedema, Lymphoma, Hodgkin's Lymphoma, Non-Hodgkin's Lymphoma, Male Breast Cancer, Malignant-Rhabdoid-Tumour-of-Kidney, Medulloblastoma, Melanoma, Merkel Cell Cancer, Mesothelioma, Metastatic Cancer, Mouth Cancer, Multiple Endocrine Neoplasia, Mycosis Fungoides, Myelodysplastic Syndromes, Myeloma, Myeloproliferative Disorders, Nasal Cancer, Nasopharyngeal Cancer, Nephroblastoma, Neuroblastoma, Neurofibromatosis, Nijmegen Breakage Syndrome, Non-Melanoma Skin Cancer, Non-Small-Cell-Lung-Cancer-(NSCLC), Ocular Cancers, Oesophageal Cancer, Oral cavity Cancer, Oropharynx Cancer, Osteosarcoma, Ostomy Ovarian Cancer, Pancreas Cancer, Paranasal Cancer, Parathyroid Cancer, Parotid Gland Cancer, Penile Cancer, Peripheral-Neuroectodermal-Tumours, Pituitary Cancer, Polycythemia vera, Prostate Cancer, Rare-cancers-and-associated-disorders, Renal Cell Carcinoma, Retinoblastoma, Rhabdomyosarcoma, Rothmund-Thomson Syndrome, Salivary Gland Cancer, Sarcoma, Schwannoma, Sezary syndrome, Skin Cancer, Small Cell Lung Cancer (SCLC), Small Intestine Cancer, Soft Tissue Sarcoma, Spinal Cord Tumours, Squamous-Cell-Carcinoma-(skin), Stomach Cancer, Synovial sarcoma, Testicular Cancer, Thymus Cancer, Thyroid Cancer, Transitional-Cell-Cancer-(bladder), Transitional-Cell-Cancer-(renal-pelvis-/-ureter), Trophoblastic Cancer, Urethral Cancer, Urinary System Cancer, Uroplakins, Uterine sarcoma, Uterus Cancer, Vaginal Cancer, Vulva Cancer, Waldenstrom's-Macroglobulinemia, Wilms' Tumour.

19. The method of claim 13, -wherein the human protein is associated with an autoimmune disease.

20. The method of claim 19, wherein the autoimmune disease is selected from the group consisting of Addisons Disease, Allergies, Anemia, Ankylosing Spondylitis, Arthritis, Celiac Disease, Crohns Disease, Diabetes, Endometriosis, Fibromyalgia, Graves Disease, Hashimotos Disease, Hypothyroidism, Immune Diseases, Lupus, Lymphoma, Meniere's Disease, Multiple Sclerosis, Oral Diseases, Osteoporosis, Pleurisy, Psoriasis, Reiters Syndrome, Rheumatoid Arthritis, Sarcoidosis, Scleroderna, Sjogrens Syndrome, Thrush, Vitiligo, Alopecia Areata, Antiphospholipid Syndrome (APS), Behcet's Disease, Ulcerative Colitis, Goodpasture Syndrome, Graft Versus Host Disease, Guillain-Barre Syndrome, Multiple Sclerosis, Myasthenia Gravis, Myositis, Pemphigus Vulgaris, Primary Biliary Cirrhosis, Rheumatic Fever, Vasculitis and Wegener's Granulomatosis.

21. A method for inducing a protective immune response against an autoimmune disease in a subject comprising administering to said subject in need of treatment a therapeutically effective amount of a composition comprising an antigen-presenting dendritic cell generated according to claim 19.

22. The method of claim 19, wherein the autoimmune disease is selected from the group consisting of Addisons Disease, Allergies, Anemia, Ankylosing Spondylitis, Arthritis, Celiac Disease, Crohns Disease, Diabetes, Endometriosis, Fibromyalgia, Graves Disease, Hashimotos Disease, Hypothyroidism, Immune Diseases, Lupus, Lymphoma, Meniere's Disease, Multiple Sclerosis, Oral Diseases, Osteoporosis, Pleurisy, Psoriasis, Reiters Syndrome, Rheumatoid Arthritis, Sarcoidosis, Scleroderma, Sjogrens Syndrome, Thrush, Vitiligo, Alopecia Areata, Antiphospholipid Syndrome (APS), Behcet's Disease, Ulcerative Colitis, Goodpasture Syndrome, Graft Versus Host Disease, Guillain-Barre Syndrome, Multiple Sclerosis, Myasthenia Gravis, Myositis, Pemphigus Vulgaris, Primary Biliary Cirrhosis, Rheumatic Fever, Vasculitis and Wegener's Granulomatosis.

23. A method for inducing a protective immune response against cancer in a subject comprising administering to said subject in need of treatment a therapeutically effective amount of a composition comprising an antigen-presenting dendritic cell generated according to claim 18.

24. A method for inducing a protective immune response against a pathogen in a subject comprising administering to said subject in need of treatment a therapeutically effective amount of a composition comprising an antigen-presenting dendritic cell generated according to any one of claims 13 to 17.
